(19) United States
(12) Patent Application Publication
(10) Pub. No.: US 2017/0349641 A1
(43) Pub. Date: Dec. 7, 2017

(54) Chiral Substituted Amyloid Beta Peptides

(71) Applicant: The Regents of the University of Calfornia, Oakland, CA (US)

(72) Inventor: Jevgenij A. Raskatov, Santa Cruz, CA (US)

(21) Appl. No.: 15/611,333

(22) Filed: Jun. 1, 2017

Related U.S. Application Data
(60) Provisional application No. 62/344,872, filed on Jun. 2, 2016.

(51) Int. Cl. C07K 14/47; G01N 33/50; G01N 33/68

(52) U.S. Cl. CPC C07K 14/4711; G01N 33/6896; G01N 33/5058; G01N 2333/4709; G01N 2500/10; G01N 2500/20; G01N 2500/04

(57) ABSTRACT

Aspects of the present disclosure include amyloid β (Aβ) peptides. In certain aspects, the Aβ peptides include a chiral substitution at an electrostatic cluster amino acid residue. Also provided are compositions, non-human animals, and kits that include the Aβ peptides. Methods involving the Aβ peptides are also provided.

### CROSS-REFERENCE TO RELATED APPLICATIONS

This application claims the benefit of U.S. Provisional Patent Application No. 62/344,872 filed Jun. 2, 2016, which application is incorporated herein by reference in its entirety.

### INTRODUCTION

Alzheimer's disease (AD) is a major neurodegenerative disorder that affects over 35 million people world-wide. Reflecting the increase in life expectancy, these numbers continue to rise, while no cure exists. Amyloid β is an aggregation prone peptide of 36-49 amino acids in length, and has been strongly implicated in the mechanism of AD. These protein aggregates are composed of mature Aβ fibrils, which represent the end product of a long and complex fibrillation process. The fibrillation pathway initiates with soluble unstructured monomeric Aβ peptides, which are converted into oligomers, protofibrils, and finally into mature fibrils.

Aβ42 is regarded as the most toxic Aβ entity in AD, which has been attributed to its high aggregation propensity. The aggregation profile is complex, with diverse oligomeric, pre-fibrillary and fibrillary states being formed. Over the past decade, diffusible oligomers have been recognized as a particularly neurotoxic species.

### SUMMARY

Aspects of the present disclosure include amyloid β (Aβ) peptides. In certain aspects, the Aβ peptides include a chiral substitution at an electrostatic cluster amino acid residue. Also provided are compositions, non-human animals, and kits that include the Aβ peptides. Methods involving the Aβ peptides are also provided.

In some embodiments, provided is a peptide including the amino acid sequence set forth in SEQ ID NO: 1 including one or more chiral substitutions at an electrostatic cluster amino acid residue selected from E22, D23, S26, K28, or any combination thereof, and where the peptide is no more than 49 amino acids in length. In certain aspects, the peptide consists of the amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14. In some embodiments, the peptide includes a chiral substitution at E22. In certain aspects, the peptide including a chiral substitution at E22 includes no other chiral substitutions. In some embodiments, a peptide of the present disclosure includes a non-chiral mutation. In certain aspects, the non-chiral mutation is selected from a non-chiral amino acid substitution, an amino acid deletion, an amino acid insertion, and any combination thereof. In some embodiments, the non-chiral mutation is a familial Aβ peptide mutation. In certain aspects, the peptide does not include a chiral substitution at amino acid residue D23, and the familial Aβ peptide mutation is a D23N mutation.

In some embodiments, provided are methods of screening a test agent for the ability to disrupt soluble oligomers of amyloid β (Aβ) peptides or the formation thereof. Such methods include incubating Aβ peptides including any of the peptides of the present disclosure under Aβ peptide aggregation conditions, contacting the Aβ peptides with a test agent, and evaluating the presence or formation of soluble oligomers of Aβ peptides to determine whether the test agent has the ability to disrupt soluble oligomers of Aβ peptides or the formation thereof. In certain aspects, the Aβ peptides are contacted with the test agent subsequent to a period during which aggregation into soluble oligomers occurs, to determine whether the test agent has the ability to disrupt soluble oligomers of Aβ peptides. In some embodiments, determining whether the test agent has the ability to disrupt soluble oligomers of Aβ peptides includes one or more of determining whether the soluble oligomers have transitioned to fibrillary aggregates, or determining whether the soluble oligomers have reverted to monomeric Aβ peptides. In certain aspects, the Aβ peptides are contacted with the test agent before or within a period during which aggregation into soluble oligomers would occur, to determine whether the test agent has the ability to disrupt the formation of soluble oligomers of Aβ peptides. In some embodiments, the test agent includes an antibody or a small molecule. In certain aspects, the methods include screening a library of test agents for the ability to disrupt soluble oligomers of Aβ peptides or the formation thereof.

Also provided are methods that include contacting a cell population with any of the peptides of the present disclosure. The methods further include, after the contacting of the cell population with the peptide, evaluating a parameter of the cell population selected from cell viability, gene expression, ubiquitin-proteosome system (UPS) function, autophagy, and any combination thereof. The methods further include contacting the cell population with a second agent, and after contacting the cell population with the second agent, evaluating a modulation by the second agent of a parameter of the cell population selected from cell viability, gene expression, ubiquitin-proteosome system (UPS) function, autophagy, and any combination thereof. In some embodiments, the second agent includes a test agent. In certain aspects, the cell population includes neuronal cells. In some embodiments, the cell population includes pheochromocytoma cells.

### BRIEF DESCRIPTION OF THE FIGURES

FIG. 1 depicts (Panel A) Hairpin region of Aβ, with the electrostatic residues E22, D23, S26 and K28 highlighted. (FIG. 1, Panels B-D) Representative foldamers of the model A21-A30 decapeptide, obtained through an NAMD molecular dynamics simulation, and displaying a D23:K28 salt bridge (Panel B), an additional stabilizing interaction with S26 (Panel C) or an alternative E22:K28 salt bridge (Panel D).

FIG. 2 depicts (Panel A) an Aβ42 sequence (SEQ ID NO: 7) with glutamate-22 highlighted in blue. The difference between Aβ40 and Aβ42 is highlighted in red. Replacement of L-glutamate with D-glutamate 22 enables for subtle alteration of the sidechain disposition. (Panel B) depicts HPLC and mass spectrum analysis of the Aβ42 wildtype (1) and E22e (2) peptide. The purity of each peptide was >95% as determined by integration of signals at 220 nm.

FIG. 3 depicts the experimental setup of the PICUP experiment with each individual component labelled.

FIG. 4 depicts (Panel A) Aggregation kinetics of 20 μM Aβ42 wild-type peptide (1) (black) and 20 μM of Aβ42 E22e peptide (2) (grey) as monitored by Thioflavin T (ThT) fluorescence (λₑₘᵢₛₛᵢₒₙ=444 nm, λ_{excitation}=485 nm) at 37° C.; (Panel B) and (Panel C) Representative transmission electron microscopy (TEM) images of the fibrillary architecture of Aβ42 wild-type peptide (1) (Panel B) and Aβ42 E22e peptide (2) (Panel C). Samples were incubated in 20 mM phosphate buffer at a concentration of 222 μM before being diluted to 200 nm for imaging.

FIG. 5 depicts the Circular Dichroism (CD) spectra of wildtype Aβ42 (1) and the E22e peptide (2). The E22e peptide shows a delay in β-sheet formation compared to the wildtype.

FIG. 6 depicts transmission electron microscopy (TEM) images of the fibrillary architecture of Aβ42 wild-type peptide (1) (Panel A, Panel C) and Aβ42 E22e peptide (2) (Panel B, Panel D). Samples were taken for imaging immediately following reconstitution (Panel A, Panel B) or following 2 h of incubation (Panel C, Panel D) at 37° C. In all instances samples were incubated in 20 mM phosphate buffer at a concentration of 222 μM before being diluted to 200 nM for imaging. Scale bar corresponds to 50 nm in all instances.

FIG. 7 depicts (Panel A) a representative PICUP gel at both t=0 h and at t=24 h. PICUP was in 20 mM phosphate buffer at a concentration of 50 μM either directly after reconstitution or following incubation in 20 mM phosphate buffer at 37° C. for 24 hours. Corresponding experiments were also performed at 20 μM; Individual replicates can be found in the supporting information; (Panel B) population states of the dimer through heptamer oligomers of the two peptides; (Panel C) SAXS of Aβ42 wild-type (1) peptide at t=0 h (black) and t=24 h (grey); (Panel D) SAXS of Aβ42 E22e (2) peptide at t=0 h (black) and t=24 h (grey). Both samples were ran in 20 mM phosphate buffer at a concentration of 222 μM.

FIG. 8 depicts the validation of the PICUP experiment for wildtype Aβ40 and Aβ42. The experimental procedure for both peptides was identical and as described in the Materials and Methods. When crosslinking was not performed (denoted by the negative symbol) only the monomer is observable (band between 3.4 and 5 kDa). Crosslinking of the solution gives rise to an oligomeric profile of both peptides.

FIG. 9 depicts the photo-induced crosslinking of unmodified wild-type Aβ42 (1) and E22e (2) at 50 and 20 μM and at either 0 h or following incubation for 24 h at 37° C. All samples were reconstituted in 20 mM phosphate buffer.

FIG. 10 depicts dose-response curves of both wild-type Aβ42 (1) (black) and Aβ42 E22e (2) (grey) peptides against the rat pheochromocytoma PC12 adhesive cell line. Cells were plated at 5000 cells per well and allowed to adhere for 24 hours prior to peptide addition, followed by incubation for an additional 72 hours. Cellular viability was determined at the end point of the assay using the reagent WST-1.

FIG. 11 depicts cytotoxicity studies of both wild-type Aβ42 (1) (grey bars) and Aβ42 E22e (2) (white) peptides against the rat pheochromocytoma PC12 adhesive cell lines. Cells were plated at 5000 cells per well and allowed to adhere for 24 hours prior to peptide addition and for a further 72 hours following dosing. Cellular viability was determined at the end point of the assay using the reagent WST-1. Three biological replicates each consisting of three technical replicates were used for analysis.

FIG. 12 depicts the effect of pre-incubation of the peptides prior to dosing to the PC12 cells. Both Aβ42 wild-type (1) and E22e peptide (2) showed comparable cytotoxicity, following either 2 h or 4 h of pre-incubation. Cells were plated at 5000 cells per well and allowed to adhere for 24 hours prior to peptide addition and for a further 72 hours following dosing. Cellular viability was determined at the end point of the assay using the reagent WST-1. Three biological replicates, each consisting of two technical replicates, were used for analysis.

### DETAILED DESCRIPTION

Provided are amyloid β (Aβ) peptides. In certain aspects, the Aβ peptides include a chiral substitution at an electrostatic cluster amino acid residue. Also provided are compositions, non-human animals, and kits that include the Aβ peptides. Methods involving the Aβ peptides are also provided.

Before the peptides, compositions, non-human animals, kits and methods of the present disclosure are described in greater detail, it is to be understood that the peptides, compositions, non-human animals, kits and methods are not limited to particular embodiments described, as such may, of course, vary. It is also to be understood that the terminology used herein is for the purpose of describing particular embodiments only, and is not intended to be limiting, since the scope of the peptides, compositions, non-human animals, kits and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the peptides, compositions, non-human animals, kits and methods. The upper and lower limits of these smaller ranges may independently be included in the smaller ranges and are also encompassed within the peptides, compositions, non-human animals, kits and methods, subject to any specifically excluded limit in the stated range. Where the stated range includes one or both of the limits, ranges excluding either or both of those included limits are also included in the peptides, compositions, non-human animals, kits and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the peptides, compositions, non-human animals, kits and methods belong. Although any peptides, compositions, non-human animals, kits and methods similar or equivalent to those described herein can also be used in the practice or testing of the peptides, compositions, non-human animals, kits and methods, representative illustrative peptides, compositions, non-human animals, kits and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present peptides, compositions, non-human animals, kits and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the peptides, compositions, non-human animals, kits and methods, which are, for clarity, described in the context of separate embodiments, may also be provided in combination in a single embodiment. Conversely, various features of the peptides, compositions, non-human animals, kits and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present peptides, compositions, non-human animals, kits and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Amyloid Beta Peptides

As summarized above, aspects of the present disclosure include Amyloid β (Aβ) peptides. In certain aspects, the Aβ peptides include a chiral substitution at an electrostatic cluster amino acid residue.

As used herein, “Amyloid β peptide” or “Aβ peptide” denotes peptides of 36 to 49 amino acids (Aβ36, Aβ37, Aβ38, Aβ39, Aβ40, Aβ41, Aβ42, Aβ43, Aβ44, Aβ45, Aβ46, Aβ47, Aβ48, and Aβ49) that are the main component of the amyloid plaques found in the brains of AD patients. The peptides result from the amyloid precursor protein (APP), which is cleaved by beta secretase and gamma secretase to yield Aβ. The amino acid sequence of the wild-type Aβ49 peptide, as well as the other wild-type Aβ peptides which are shorter versions of the Aβ49 peptide, are provided in Table 1 below.

TABLE 1
Wild-type Aβ peptide amino acid sequences

| | |
|---|---|
| Aβ36 | DAEFRHDSGYEVHHQKLVFFAEDVGSNKGAIIGLMV |
| SEQ ID | |
| NO: 1 | |
| Aβ37 | DAEFRHDSGYEVHHQKLVFFAEDVGSNKGAIIGLMVG |
| SEQ ID | |
| NO: 2 | |
| Aβ38 | DAEFRHDSGYEVHHQKLVFFAEDVGSNKGAIIGLMVGG |
| SEQ ID | |
| NO: 3 | |
| Aβ39 | DAEFRHDSGYEVHHQKLVFFAEDVGSNKGAIIGLMVGGV |
| SEQ ID | |
| NO: 4 | |
| Aβ40 | DAEFRHDSGYEVHHQKLVFFAEDVGSNKGAIIGLMVGGVV |
| SEQ ID | |
| NO: 5 | |
| Aβ41 | DAEFRHDSGYEVHHQKLVFFAEDVGSNKGAIIGLMVGGVVI |
| SEQ ID | |
| NO: 6 | |
| Aβ42 | DAEFRHDSGYEVHHQKLVFFAEDVGSNKGAIIGLMVGGVVI |
| SEQ ID | A |
| NO: 7 | |
| Aβ43 | DAEFRHDSGYEVHHQKLVFFAEDVGSNKGAIIGLMVGGVVI |
| SEQ ID | AT |
| NO: 8 | |
| Aβ44 | DAEFRHDSGYEVHHQKLVFFAEDVGSNKGAIIGLMVGGVVI |
| SEQ ID | ATV |
| NO: 9 | |
| Aβ45 | DAEFRHDSGYEVHHQKLVFFAEDVGSNKGAIIGLMVGGVVI |
| SEQ ID | ATVI |
| NO: 10 | |
| Aβ46 | DAEFRHDSGYEVHHQKLVFFAEDVGSNKGAIIGLMVGGVVI |
| SEQ ID | ATVIV |
| NO: 11 | |
| Aβ47 | DAEFRHDSGYEVHHQKLVFFAEDVGSNKGAIIGLMVGGVVI |
| SEQ ID | ATVIVI |
| NO: 12 | |
| Aβ48 | DAEFRHDSGYEVHHQKLVFFAEDVGSNKGAIIGLMVGGVVI |
| SEQ ID | ATVIVIT |
| NO: 13 | |
| Aβ49 | DAEFRHDSGYEVHHQKLVFFAEDVGSNKGAIIGLMVGGVVI |
| SEQ ID | ATVIVITL |
| NO: 14 | |

The Aβ peptide having one or more chiral substitutions may be an Aβ36 peptide, an Aβ37 peptide, an Aβ38 peptide, an Aβ39 peptide, an Aβ40 peptide, an Aβ41 peptide, an Aβ42 peptide, an Aβ43 peptide, an Aβ44 peptide, an Aβ45 peptide, an Aβ46 peptide, an Aβ47 peptide, an Aβ48 peptide, or an Aβ49 peptide.

As summarized above, the Aβ peptides of the present disclosure include a chiral substitution at an electrostatic cluster amino acid residue. By “chiral substitution” is meant an L form of an amino acid is substituted with a D form of that amino acid. For example, an Aβ peptide having a chiral substitution at E22 (the glutamate residue at position 22) has the D form of glutamate at residue 22 rather than the L form. Only L-amino acids are manufactured in cells and incorporated into proteins. Glycine, the simplest amino acid, has no enantiomers because it has two hydrogen atoms attached to the central carbon atom. Only when all four attachments are different can enantiomers occur.

By “electrostatic cluster amino acid residue” is meant an amino acid residue present in the Aβ peptide electrostatic cluster. The central region of Aβ encompasses the amino acids E22, D23, S26 and K28, which can be thought of as an electrostatic cluster within which the following interactions are possible:

1. E22:D23—charge-charge repulsion

2. E22:S26—charge-dipole attraction

3. D23:S26—charge-dipole attraction

4. E22:K28—charge-charge attraction

5. D23:K28—charge-charge attraction

6. S26:K28—charge-charge attraction

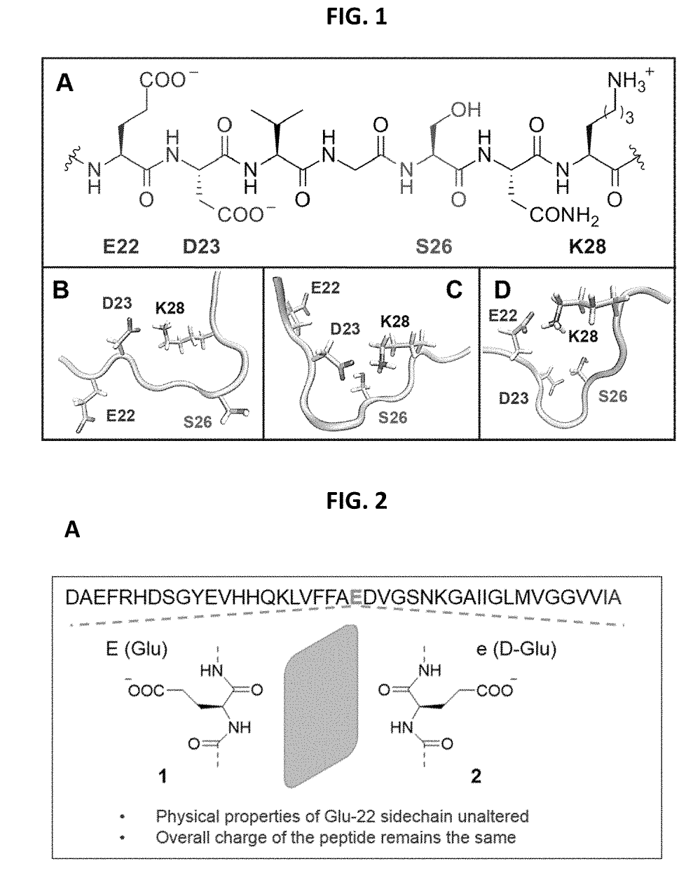

FIG. 1, panel A, shows the hairpin region of Aβ that includes electrostatic residues E22, D23, S26 and K28. Also shown in FIG. 1 are representative foldamers of the model A21-A30 decapeptide, obtained through an NAMD molecular dynamics simulation, and displaying a D23:K28 salt bridge (Panel B), an additional stabilizing interaction with S26 (Panel C) or an alternative E22:K28 salt bridge (Panel D). According to certain embodiments, the Aβ peptide includes one or more chiral substitutions at an electrostatic cluster amino acid residue selected from E22, D23, S26, K28, and any combination thereof. As shown in the Table 2 below, independent chiral substitutions of these four residues results in 16 possible variants, any of which may be encompassed by the Aβ peptides of the present disclosure.

TABLE 2
16 Independent chirality permutations (E22:D23:S26:K28)

| | Aβ42 Variant | | | | | | | | | | | | | | | |
|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 |
| E22 | L | D | L | L | L | D | D | D | L | L | L | D | D | D | L | D |
| D23 | L | L | D | L | L | D | L | L | D | D | L | D | D | L | D | D |
| S26 | L | L | L | D | L | L | D | L | D | L | D | D | L | D | D | D |
| K28 | L | L | L | L | D | L | L | D | L | D | D | L | D | D | D | D |

In certain aspects, the Aβ peptide (e.g., an Aβ42 peptide or an Aβ40 peptide), includes a chiral substitution at amino acid residue E22. The chiral substitution at amino acid residue E22 may be the only chiral substitution in the peptide, or may be one of two or more chiral substitutions in the peptide.

According to certain embodiments, the Aβ peptide further includes a mutation (e.g., a non-chiral mutation) that is not a chiral substitution at an electrostatic cluster amino acid residue. For example, the Aβ peptide having a chiral substitution at an electrostatic cluster amino acid residue may further include one or more non-chiral mutations selected from a non-chiral amino acid substitution, an amino acid deletion, an amino acid insertion, and any combination thereof.

In certain aspects, the Aβ peptide includes one or more familial Aβ peptide mutations. Familial AD can arise from diverse mutations within the Aβ (e.g., Aβ42) sequence. Over ten Aβ42 mutations have been identified, most of which are disease-causing single amino acid alterations. Four AD-accelerating variants include mutations involving the E22 residue, and the mutations alter the charge at that residue—either through amino acid substitution (E22G, Arctic; E22K, Italian; E22Q, Dutch) or through amino acid deletion (E22A, Osaka). Biophysical experiments demonstrated those substitutions to enhance Aβ propensity towards oligomer, or fibril formation. Other familial mutations include, e.g., D23N (Iowa).

According to certain embodiments, the Aβ peptide does not include a chiral substitution at amino acid residue E22, and the familial Aβ peptide mutation is selected from an E22G mutation, an E22K mutation, an E22Q mutation, or an E22Δ mutation. In certain aspects, the Aβ peptide does not include a chiral substitution at amino acid residue D23, and the familial Aβ peptide mutation is a D23N mutation.

Methods of Making Aβ Peptides

Methods of making the Aβ peptides of the present disclosure are also provided. In certain aspects, the peptide is produced using a chemical peptide synthesis technique. Where a peptide is chemically synthesized, the synthesis may proceed via liquid-phase or solid-phase. Solid phase peptide synthesis (SPPS), in which the C-terminal amino acid of the sequence may be attached to an insoluble support followed by sequential addition of the remaining amino acids in the sequence (including the relevant D-amino acid(s) at one or more electrostatic cluster amino acid residue positions), is an example of a suitable method for the chemical synthesis of a peptide of the present disclosure. Various forms of SPPS, such as Fmoc and Boc, are available for synthesizing the peptide. For example, small insoluble, porous beads may be treated with functional units on which peptide chains are built. After repeated cycling of coupling/deprotection, the free N-terminal amine of a solid-phase attached is coupled to a single N-protected amino acid unit. This unit is then deprotected, revealing a new N-terminal amine to which a further amino acid may be attached. The peptide remains immobilized on the solid-phase and may undergo a filtration process before being cleaved off.

One example of a suitable strategy for synthesizing an Aβ peptide of the present disclosure is provided in the Experimental section below.

Once synthesized, the peptide can be purified according to standard procedures, including ammonium sulfate precipitation, affinity columns, column chromatography, high performance liquid chromatography (HPLC) purification, gel electrophoresis, and the like.

Aβ Peptide Compositions

Also provided are compositions that include the Aβ peptides of the present disclosure. In certain aspects, the composition includes any of the Aβ peptides of the present disclosure present in a liquid medium. The liquid medium may be an aqueous liquid medium, such as water, a buffered solution (e.g., a phosphate buffered solution), or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a protease inhibitor, glycerol, a chelating agent, and the like may be present in such compositions.

According to some embodiments, the composition includes the Aβ peptide present in lyophilized form. Such compositions may include one or more stabilizing components, such as a sugar alcohol. Sugar alcohols are known to stabilize macromolecules when freeze-dried or in a liquid state, and to prevent denaturation. An example of sugar alcohol that may be present (e.g., as a lyoprotectant) in a lyophilized composition of the present disclosure includes, but is not limited to, cyclodextrin, mannitol, sorbitol, glycerol, xylitol, inositol, or the like. The lyophilized compositions of the present disclosure may be storage stable, that is, maintained for an extended period of time.

Non-Human Animals Including Aβ Peptides

Also provided are non-human animals that include the Aβ peptides of the present disclosure. Such non-human animals find use, e.g., for conducting in vivo Aβ peptide aggregation and/or cytotoxicity studies. In certain aspects, the non-human animal is a mammal. In certain aspects, the mammal is selected from a rodent, a non-human primate, a pig, a canine, a feline, a sheep, a goat, a cow, and a horse.

A non-human animal of the present disclosure may be generated by administering to a non-human animal any of the Aβ peptides of the present disclosure. Administration may be local or systemic. According to certain embodiments, administration is by a topical, enteral (e.g., oral), or parenteral route of administration. Parenteral routes of administration of interest include, but are not limited to, intracerebral injection (e.g., injection into the brain parenchyma), intracerebroventricular injection, intrathecal injection, intravenous injection, and intra-arterial injection. In certain aspects, the Aβ peptide is formulated for injection by dissolving, suspending or emulsifying the Aβ peptide in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Suitable compositions that include the Aβ peptide may be prepared by mixing the Aβ peptide having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

Formulations of the Aβ peptides of the present disclosure suitable for administration to a non-human animal are generally sterile and may further be free of detectable pyrogens or other contaminants contraindicated for administration to an animal according to a selected route of administration.

According to certain embodiments, the non-human animals of the present disclosure include a transgene (that is, are transgenic). The transgene is present in a transgenic construct configured to express in the non-human animal a gene of interest or modulate the expression of a gene of interest, e.g., by expressing an siRNA. In certain aspects, the gene of interest is known to be involved in Aβ peptide aggregation and/or cytotoxicity. The transgene may be operably linked to a ubiquitous promoter, a tissue-specific promoter, an inducible promoter, and/or the like. According to certain embodiments, the transgene encodes the amyloid precursor protein (APP). For example, the non-human animal that includes an Aβ peptide of the present disclosure may be a mouse of the J20 mouse line which overexpresses human APP with two mutations linked to familial Alzheimer's disease (the Swedish and Indiana mutations).

In certain aspects, in addition to including an Aβ peptide of the present disclosure, the non-human animal includes a knock-out mutation, e.g., achieved via homologous recombination in ES cells. For example, the non-human animal may lack a gene with a known involvement in Aβ peptide aggregation and/or cytotoxicity.

Kits

Also provided are kits that include the Aβ peptides of the present disclosure. In certain aspects, the kits find use in the formation and study of soluble oligomers of Aβ. For example, an Aβ peptide of the subject kits may be used to generate soluble oligomers of Aβ to facilitate screening for agents (e.g., small molecules, antibodies, and/or the like) that disrupt the soluble oligomers of Aβ.

In certain aspects, in addition to the Aβ peptide, the kits include one or more reagents that find use in using the Aβ peptide in an Aβ peptide aggregation assay. Exemplary reagents include, e.g., Thioflavin T (TFT) and/or any other useful reagents, such as those described in the Experimental section below.

Components of the kits may be present in separate containers, or multiple components may be present in a single container. For example, the Aβ peptide and any other optional reagents, etc. may be provided in the same tube, or may be provided in different tubes.

In addition to the above-mentioned components, a subject kit may further include instructions for using the components of the kit, e.g., to practice the subject methods. For example, the kits may include instructions for using the Aβ peptide in an Aβ peptide aggregation assay. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, Hard Disk Drive (HDD) etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Methods

As summarized above, aspects of the present disclosure include methods that employ the Aβ peptides of the present disclosure.

According to some embodiments, a method of the present disclosure includes providing a container that includes any of the Aβ peptides described herein, and evaluating Aβ aggregation (e.g., including aggregation of the Aβ peptide) in the container. Conditions suitable for Aβ peptide aggregation are known, and example conditions are described in the Experimental section below. For example, aggregation conditions involving the use of Thioflavin T (ThT) may be employed to facilitate Aβ peptide aggregation.

Containers that find use in performing the methods of the present disclosure include, e.g., a tube, a well (e.g., a well of a multi-well plate), or the like.

By “evaluating Aβ aggregation” or “evaluating aggregation of the Aβ peptide” is meant evaluating the aggregation process (e.g., the aggregation rate and/or the like) and/or evaluating the structure of the aggregates (e.g., soluble oligomers of Aβ) produced as a result of the aggregation. For example, in certain aspects, evaluating aggregation of the Aβ peptide includes imaging the Aβ aggregates, e.g., to examine structural features of the aggregates. The imaging approach may vary depending on the type of structural information desired. According to certain embodiments, imaging the Aβ aggregates includes obtaining one or more electron microscopy images (e.g., transmission electron microscopy (TEM) images) of the aggregates.

In certain aspects, evaluating aggregation of the Aβ peptide includes obtaining spectroscopic data of aggregates of the Aβ peptide. In certain embodiments, the spectroscopic data includes time-resolved circular dichroism spectra of the peptides. Such spectra find use, e.g., in evaluating random coil to β-sheet configuration of the peptides/aggregates.

According to certain embodiments, evaluating aggregation of the Aβ peptide includes analyzing aggregates of the Aβ peptide by small angle X-ray scattering (SAXS). SAXS is capable of revealing solution structures of biological molecules (e.g., peptides) at 1-2 nm resolution. The SAXS analysis may involve illuminating a sample using X-rays and registering the scattered radiation using a detector. SAXS measurements are done very close to the primary beam, hence “small angles”. According to certain embodiments, analyzing aggregates of the Aβ peptide by SAXS includes determining the presence or absence of a Bragg reflection.

In certain aspects, evaluating aggregation of the Aβ peptide includes evaluating aggregation kinetics of the Aβ peptide. For example, the methods may include determining the rate of fibril formation.

According to certain embodiments, the container that includes the Aβ peptide further includes an agent, and evaluating aggregation of the Aβ peptide includes evaluating an effect of the agent on aggregation of the Aβ peptide, e.g., aggregation kinetics, aggregate morphology, and/or the like. Evaluating an effect of the agent may include evaluating aggregation of the Aβ peptide in the absence of the agent and comparing the aggregation to the aggregation of the Aβ peptide in the presence of the agent. Such aspects of the methods find use, e.g., in identifying agents (e.g., small molecules, antibodies, etc.) that disrupt soluble oligomers (e.g., highly cytotoxic soluble oligomer intermediates) of certain Aβ peptides of the present disclosure.

According to certain embodiments, the container that includes the Aβ peptide further includes a population of cells, e.g., neuronal cells, such as pheochromocytoma (PC12) cells. The cells may be in suspension or adhered to a surface of the container.

Also provided are methods that include contacting a cell population with any of the Aβ peptides of the present disclosure. In certain aspects, the cells are present in a cell culture dish or well, and a medium (e.g., a cell culture medium) that includes the Aβ peptide is added to the dish or well. The contacting may be for a sufficient period of time for aggregation of the Aβ peptide to occur. The methods may further include evaluating a parameter selected from cell viability, gene expression, ubiquitin-proteosome system (UPS) function, autophagy, and any combination thereof.

Cell viability may be determined by dosing adherent cells (e.g., adherent pheochromocytoma (PC12) cells) with media containing the Aβ peptide, incubating the cells for a sufficient period of time (e.g., 72 hours), adding a cell proliferation reagent (e.g., WST-1), and measuring a suitable parameter, e.g., absorbance at λ=490 nm.

Gene expression in the cells contacted with the Aβ peptide may be evaluated using any of a variety of approaches, including real-time RT-PCR to detect/quantitate RNA transcripts of the interest, microarray approaches, and the like. In certain aspects, transcriptome-wide profiling of gene expression changes in response to treatment with the Aβ peptide is performed.

Dysfunctions in the ubiquitin-proteasome system (UPS) are associated with the pathogenesis of Alzheimer's disease. Mutations in ubiquitin and ubiquilin-1 lead to deficits in the UPS and lead to reduced clearance of Aβ. Toxic aggregation states of Aβ inhibit the UPS by interacting with the components of the system, leading to inefficient protein degradation and dysfunctions in cellular quality control. Another protein clearance pathway that is known to contribute to Alzheimer's disease pathogenesis is the autophagy pathway. Accordingly, in some embodiments, the methods include assessing changes in the UPS, autophagy, or both, in response to treatment of the cells with an Aβ peptide of the present disclosure.

In certain aspects, the methods that include contacting a cell population with an Aβ peptide of the present disclosure further include contacting the cell population with a second agent (where the Aβ peptide may be regarded as the “first agent”). The second agent may be provided prior to, concurrently with, or subsequent to the contacting of the cells with the Aβ peptide. According to certain embodiments, after contacting the cell population with the second agent, the methods further include evaluating a modulation by the second agent of a parameter selected from cell viability, gene expression, ubiquitin-proteosome system (UPS) function, autophagy, and any combination thereof.

Any cell population of interest may be contacted with the Aβ peptide (and optionally, a second agent). For example, the cells may be neuronal cells. Neuronal cells of interest include, but are not limited to, pheochromocytoma (PC12) cells.

Also provided are methods of screening a test agent for the ability to disrupt soluble oligomers of Aβ peptides or the formation thereof. Such methods include incubating Aβ peptides including any of the Aβ peptides of the present disclosure under Aβ peptide aggregation conditions, contacting the Aβ peptides with a test agent, and evaluating the presence or formation of soluble oligomers of Aβ peptides to determine whether the test agent has the ability to disrupt soluble oligomers of Aβ peptides or the formation thereof. In certain aspects, the Aβ peptides are contacted with the test agent subsequent to a period during which aggregation into soluble oligomers occurs, to determine whether the test agent has the ability to disrupt existing soluble oligomers of Aβ peptides. Determining whether the test agent has the ability to disrupt soluble oligomers of Aβ peptides may include determining whether the soluble oligomers have transitioned to fibrillary aggregates (e.g., protofibrils, mature fibrils, and/or plaques). According to certain embodiments, determining whether the test agent has the ability to disrupt soluble oligomers of Aβ peptides includes determining whether the soluble oligomers have reverted to monomeric Aβ peptides.

According to certain embodiments, the screening methods of the present disclosure include contacting the Aβ peptides with the test agent before or within a period during which aggregation into soluble oligomers would occur, to determine whether the test agent has the ability to disrupt the formation of soluble oligomers of Aβ peptides.

The type of test agent employed in the screening methods of the present disclosure may vary. In certain aspects, the test agent is a small molecule. By “small molecule” is meant a compound having a molecular weight of 1000 atomic mass units (amu) or less. In some embodiments, the small molecule is 750 amu or less, 500 amu or less, 400 amu or less, 300 amu or less, or 200 amu or less. In certain aspects, the small molecule is not made of repeating molecular units such as are present in a polymer.

According to certain embodiments, the test agent is an antibody. The terms “antibody” and “immunoglobulin” include antibodies or immunoglobulins of any isotype (e.g., IgG, IgE, IgD, IgA, IgM, etc.), whole antibodies (e.g., antibodies composed of a tetramer which in turn is composed of two dimers of a heavy and light chain polypeptide); single chain antibodies; fragments of antibodies (e.g., fragments of whole or single chain antibodies) which retain specific binding to the cell surface molecule of the target cell, including, but not limited to single chain Fv (scFv), Fab, (Fab′)2, (scFv′)2, and diabodies; chimeric antibodies; monoclonal antibodies, human antibodies, humanized antibodies (e.g., humanized whole antibodies, humanized half antibodies, or humanized antibody fragments); and fusion proteins including an antigen-binding portion of an antibody and a non-antibody protein. The antibodies may be detectably labeled, e.g., with a fluorescent label, a radioisotope, an enzyme which generates a detectable product, or the like. The antibodies may be further conjugated to other moieties, such as members of specific binding pairs, e.g., biotin (member of biotin-avidin specific binding pair), and the like.

The test agent may be part of a library of test agents, e.g., a drug screening library, and the methods may include screening a library of test agents for the ability to disrupt soluble oligomers of Aβ peptides or the formation thereof. Combinatorial library techniques facilitate the synthesis of many molecules. These techniques can be combined with high throughput screening (HTS) to screen many compounds (agents) to identify agents capable of disrupting soluble oligomers of Aβ peptides or the formation thereof. Small molecule compound libraries for drug discovery and drug screening are commercially available, e.g., from Selleckchem, Sigma-Aldrich (St. Louis, Mo.), and elsewhere.

The following examples are offered by way of illustration and not by way of limitation.

### EXPERIMENTAL

### Materials and Methods

### Aβ Synthesis, Purification and Reconstitution:

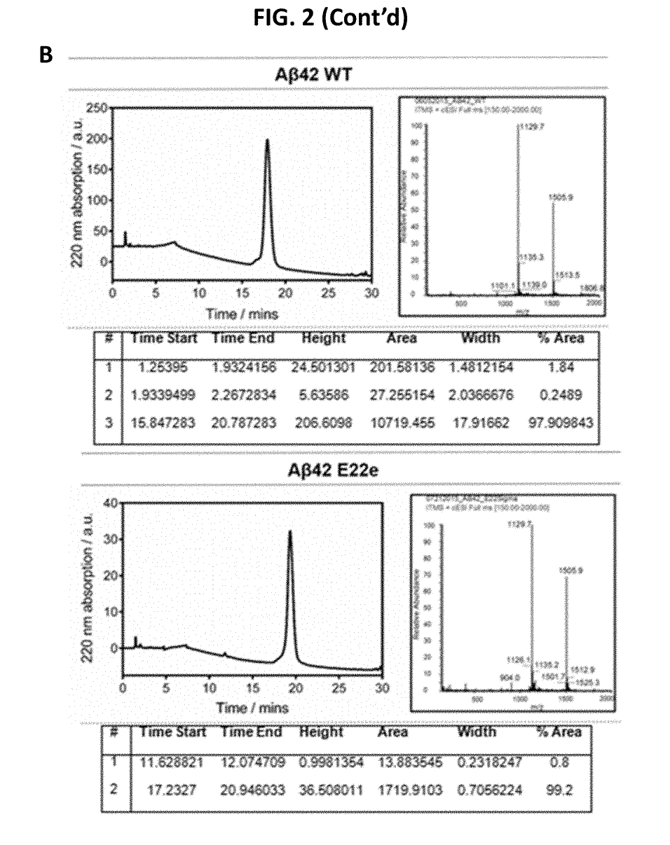

Compounds (1) and (2) (see, FIG. 2) were synthesized on HMPB ChemMatrix resin, pre-loaded with L-Ala, employing standard Fmoc coupling chemistry conditions. Cleavage under acidic conditions with subsequent ether precipitation was followed by HPLC purification, employing a PLRP-S column (8 μm, 300 Å) under basic conditions, as recommended by Agilent to attenuate on-column aggregation. The aqueous buffer (A) consisted of 20 mM ammonium hydroxide, whereas the organic buffer was composed of 80% acetonitrile and 20% 20 mM aqueous ammonium hydroxide. Following existing protocols, samples were lyophilized and taken up in 20 mM NaOH, adjusted to represent 5% of the final volume, and diluted with either phosphate buffer or PBS, depending on the nature of the experiment (final pH was 7.4 in all cases).

### Thioflavin T Aggregation Studies:

Preparation of Thioflavin T solution: A ThT solution was freshly prepared before use. 4 mg of ThT was dissolved in 10 mL of commercially bought 1×-PBS buffer (pH 7.4, 10 mM) containing 0.02% (w/v) NaN₃. The solution was filtered through a 0.22 micron filter and the concentration of a 1/20 dilution of the filtrated solution was determined by Nanodrop (ε=36000M⁻¹ cm⁻¹ at 412 nm). An aliquot of this stock solution was combined with 1×-PBS (10 mM, pH 7.4) containing 0.02% NaN₃ (w/v) to obtain a 100 μM ThT stock solution. Preparation of Aβ42 solution: 0.5 mg of 0.1% NH₄OH pretreated Aβ42 WT (1) or E22e (2) were freshly dissolved separately in 50 μL of 20 mM NaOH solution. The mixtures were sonicated for 30 s. Aβ42 solutions were then diluted by adding 450 μL of cold 1×-PBS (10 mM, pH 7.4) and filtered through 100 kDa MWCO spin filter (Corning® Spin-XR UF 500, catalog number: 431481) at 4° C. at 16200 rcf for 5 min. The filter was washed three times with cold 1×-PBS (pH 7.4) before use. The concentration of filtered Aβ42 solutions was measured by Nanodrop (ε=1490 M⁻¹ cm⁻¹ at 280 nm).

Preparation of ThT experiments: All experiments were conducted in black, clear bottom 96-well plates with shaking in a Molecular Device Gemini EM fluorescence plate reader (λem=444 nm, λex=485 nm) at 37° C. Each well contained 200 μL of reaction solution containing 20 μM of peptide 1 or 2, 20 μM ThT and 1×-PBS buffer (10 mM, pH 7.4) in presence of 0.02% (w/v) NaN₃. The wells were prepared in quintuplicate or greater and the plate was sealed with optically clear adhesive film. Each ThT assay was conducted for 2 hours at 37° C. Readings were collected every 5 min with 5 s shaking before reading and 295 s shaking in between readings.

### Circular Dichroism Studies:

Circular dichroism experiments were performed on a Jasco 1500 circular dichroism spectrometer set to a scan range of 180 to 280 nm, a DIT of 4 seconds and a scan speed of 50 nm per minute. For experiments involving wildtype Aβ42 1, 90 μg of NH4OH pre-treated protein was dissolved in 20 μI of an aqueous solution of 20 mM NaOH and sonicated for 30 seconds. 380 μI of freshly prepared 20 mM phosphate buffer solution at a pH 7.4 not containing sodium chloride was then added resulting in a final concentration of 50 μM and final volume of 400 μI. The solution was immediately transferred to the cuvette and CD analysis undertaken. Following the initial analysis, the solution was removed from the cuvette and transferred to a low-bind 1.5 ml eppendorf tube and incubated in a mini-dry bath at 37° C. for the appropriate time. This procedure was repeated for all time points collected. For the E22e (2) peptide the same procedure as described above was applied.

### Photochemically Induced Crosslinking of Unmodified Proteins (PICUP):

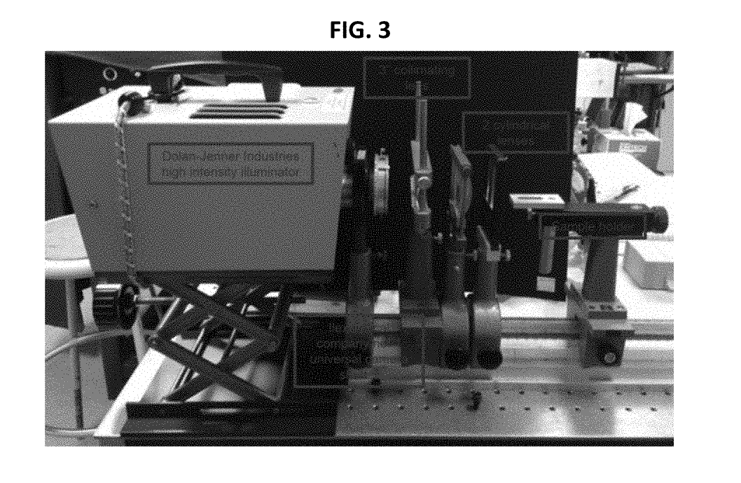

4 μL of 1 mM [Ru(bipy)₃]²⁺ and 4 μL of 20 mM ammonium persulfate were added to 32 μL of either a 50 μM or 20 μM solution of the reconstituted peptide in 20 mM phosphate buffer. The sample was irradiated for 1 s using the setup as shown in FIG. 3. Following irradiation the solution was immediately quenched with 40 μL of loading buffer containing 5% 2-mercaptoethanol. The oligomeric distribution of the peptide was determined by SDS-page gel electrophoresis using a 12% tris-tricine polyacrylamide gel. The voltage of the system was kept constant at 100V for 2 hours. Following electrophoresis the gels were silver stained and the oligomeric bands analyzed.

### Transmission Electron Microscopy (TEM) of Wild-Type (1) and E22e (2):

Aβ42 wild-type (1) or Aβ42 E22e (2) fibrils were grown for 0 h, 2 h or 7 days at 37° C. in 20 mM phosphate buffer (pH 7.4) at a concentration of 222 μM. At the appropriate time point, 1 μI of the fibril sample was added to 999 μI of milliQ water in order to obtain an imaging concentration of 200 nM. 3 μI aliquots of this material were adsorbed to activated carbon-coated Formvar 200 mesh copper grids for 1 minute and then negatively stained with 30 μI 1% (w/v) uranyl acetate. All fibrils were imaged by using FEI Tecnai 12 microscope at 120 kV.

### Small Angle X-ray Scattering (SAXS) of Wild-Type (1) and E22e (2):

Small angle x-ray scattering was collected at the SIBYLS beamline at the Advanced Light Source (Berkeley, Calif.). All samples were ran at a concentration of 1 mg/ml (222 μM). Samples were exposed to the beamline for either 0.5, 1, 2 or 4 seconds. Samples were placed 1.5 m from a MAR165 CCD detector arranged coaxial with the 12 keV monochromatic beam; 1012 photons/second were impingent on the sample. Scattering data were plotted on log of x-ray intensity scale versus momentum transfer (q) in inverse Å, where q=(4π sin(θ/2))/λ, θ is the scattering angle relative to the incident beam, and λ is the wavelength.

### Cytotoxicity Analysis of Peptides Against the Rat Pheochromocytoma (PC12) Cell Line:

Adhesive PC12 cells were purchased from ATCC (1721.1) and cultured in accordance with the supplier's instructions. For cytotoxicity experiments, cells were plated at a density of 5000 cells/well and incubated for 24 hours before dosing. Each well contained 100 μI total volume. For dosing, 100 μg of 0.1% NH₄OH pre-treated peptide was dissolved in 20 μI of 20 mM NaOH. The solution was diluted to a final concentration of 20 μM using 980 μI of F12-K media containing 2.5% FBS and 15% HS. 500 μI of this solution was removed to allow for serial dilution of the peptide concentration. Following 72 hour incubation, 10 μI of WST-1 (Roche) was added to each well and incubated for a further 2 hours. The absorbance at λ=490 nm was used to determine cellular viability. For pre-incubation experiments the same protocol was used, except that samples were pre-incubated for indicated time periods in the cell culture media before dosing.

### EXAMPLE 1

### Effect of the E22e Mutant on Aggregation of Aβ42

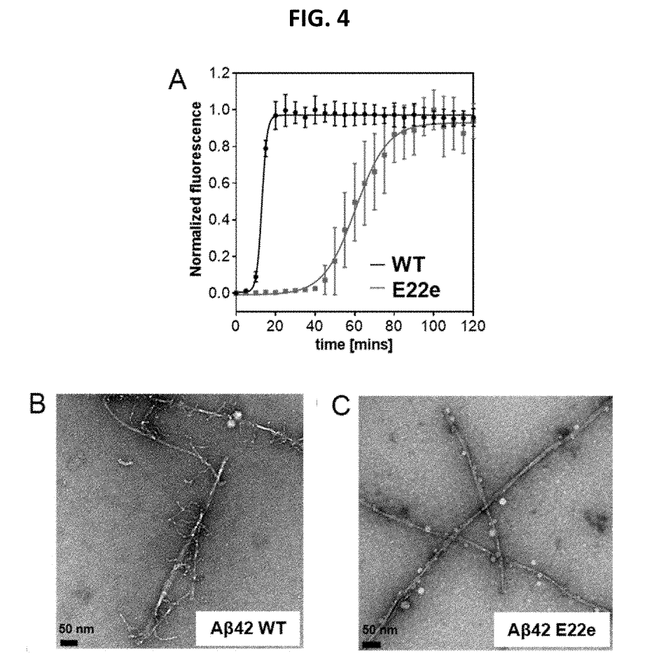

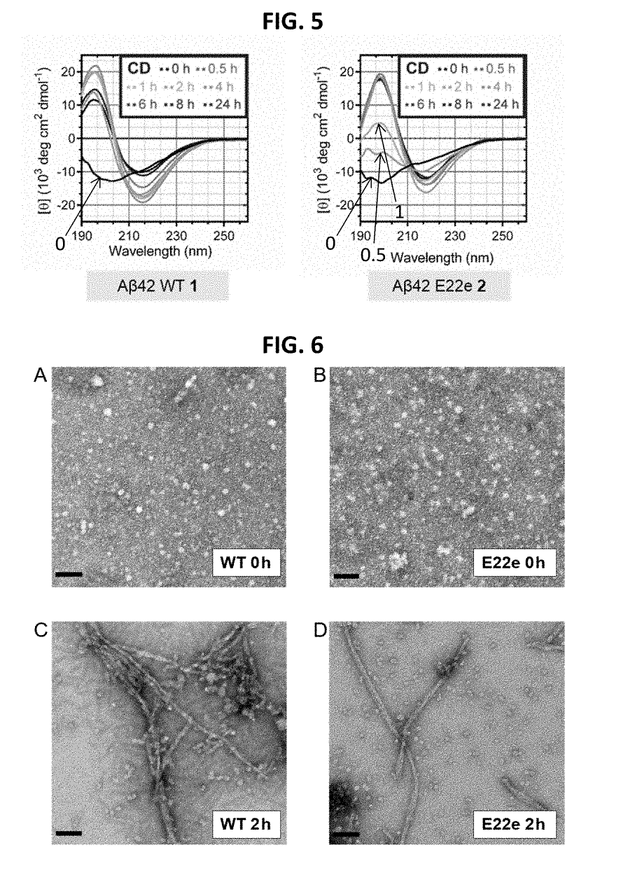

The effect of introduction of D-glutamate at position 22 on the aggregation propensity of Aβ42 was studied by conducting Thioflavin T experiments (FIG. 4, Panel A). The chiral E22e mutant (2) exhibited a fivefold reduction in fibril formation rate, as compared against the wildtype peptide (1) (t_{1/2}, E22e=65.6 min vs t_{1/2}, WT=13.4 min). The rate of fibril formation of (1) was comparable to those previously reported in the literature. The ability of the Aβ42 peptide to aggregate may stem from its propensity to undertake a secondary structural transition from a random coil-like structure through to a β-sheet configuration. Therefore, the time-resolved circular dichroism spectra of the peptides (1) and (2) was examined over a period of 24 hours. In agreement with thioflavin T results, a delay in random coil to β-sheet configuration of the Aβ E22e peptide (2) was observed (FIG. 5). These results demonstrate a reduced propensity for aggregation of peptide (2) at the fibrillary end-point, as well as at pre-fibrillary stages.

### EXAMPLE 2

### Effect of the E22e Mutant on Fibrillary Morphology

The delayed aggregation kinetics of the E22e peptide (2) led to the investigation of whether the fibrillary assemblies of the peptide (2) were altered, when compared to (1). To do this, both Aβ42 WT 1 and Aβ42 E22e 2 fibrils were grown for 7 days at 37° C. following known protocols (also see, Materials and Methods). As can be seen in FIG. 4, Panel B, transmission electron microscopy (TEM) of wild-type Aβ42 fibrils showed a distinct fibrillary architecture, characterized by the presence of numerous branches extending from the main fibril. In contrast, peptide (2) displayed more elongated, organized fibrillary structures devoid of branches that were observed for wildtype peptide (1) (FIG. 4, Panel C). Analogous TEM experiments were conducted, following an incubation of the peptides (1) and (2) for 2 h. Results were consistent in terms of branching, which was observable for Aβ42 WT, but not the E22e chiral variant (FIG. 6).

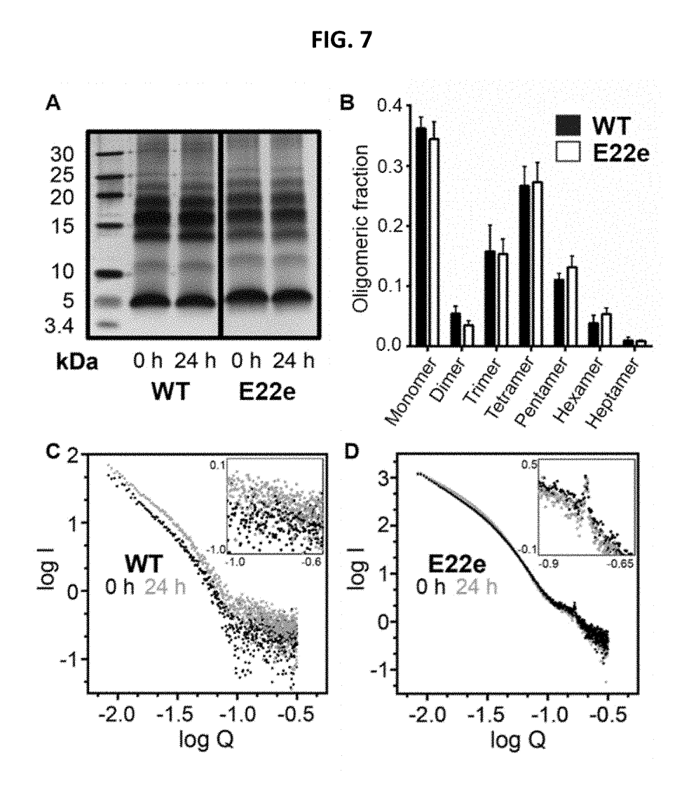

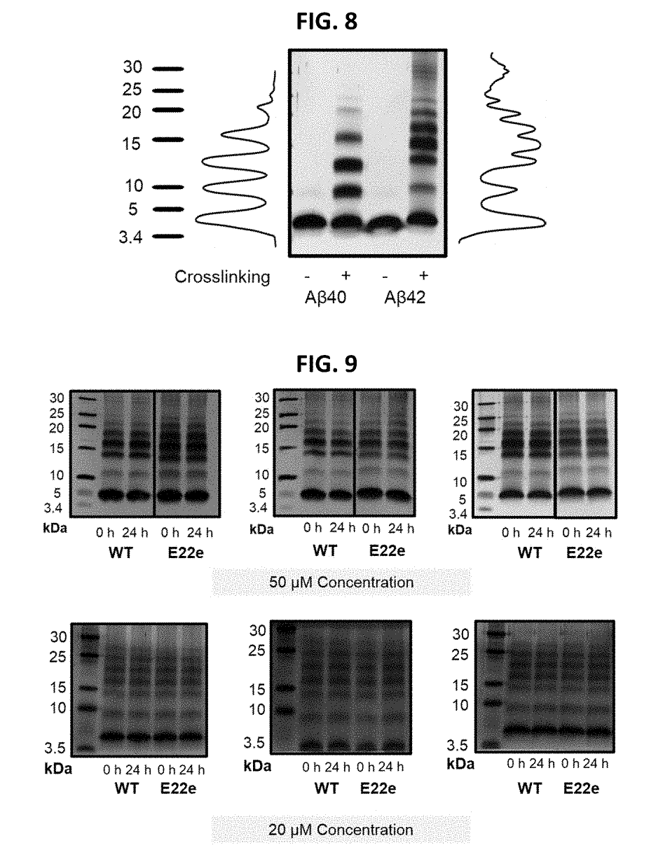

The difference in the fibrillary morphologies between the peptides (1) and (2) led to the further investigation of whether differences in the pre-fibrillary structural assemblies could account for the striking differences in fibril morphology. Photochemically induced crosslinking of unmodified proteins (PICUP) experiments were carried out to gain insight into the distribution of oligomeric states. Comparative analyses of wild-type Aβ42 (1) and the E22e peptide (2) were conducted at two time points, either immediately upon reconstitution or following an incubation for 24 h. The oligomerization profiles of the two scaffolds (1) and (2) showed no statistically significant difference in the population states of the dimer through heptamer oligomers indicating that any differences in the fibrillary assembly of the two peptides occurred at more advanced stages of aggregation. (FIG. 7 (Panels A and B), FIG. 8 and FIG. 9). To probe these late stage pre-fibrillary structures, small angle X-ray scattering (SAXS) analysis was performed. SAXS has been shown to be a powerful technique for monitoring amyloid-related structural features. The SAXS curves of both Aβ42 wild-type (1) and E22e peptide (2) were examined after initial reconstitution and following 24 hour incubation at 37° C. (FIG. 7, Panels C and D). For both time points, SAXS analysis of peptide (2) demonstrated a Bragg reflection corresponding to a species with a periodicity of 3.7 nm. This value is consistent with the dimensions of a single unit of wild-type Aβ42 found within a fibril using NMR and in silico structural models. No Bragg reflection and an increase in heterogeneity of the sample was observed for peptide (1) as reflected by the large variance at high Q values.

### EXAMPLE 3

### Effect of the E22e Mutant on Cytotoxicity

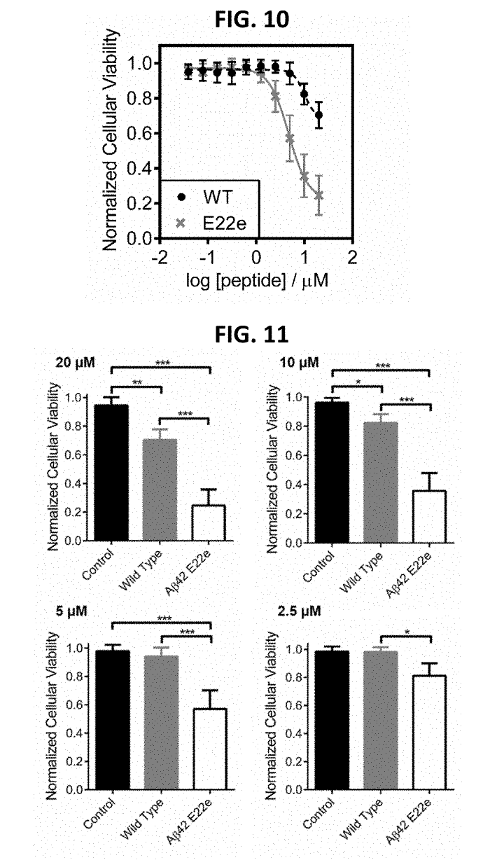

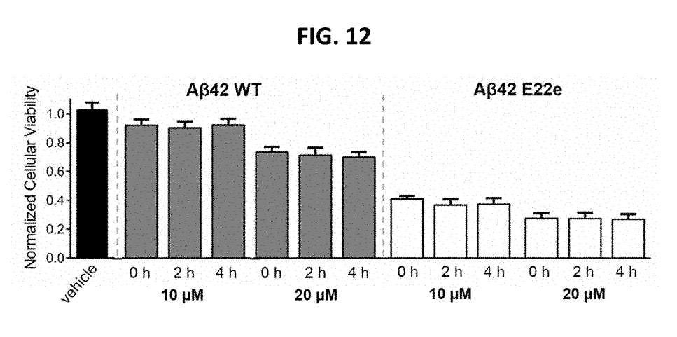

Diverse modes of cytotoxicity have been proposed, including membrane disruption, induction of τ-hyperphosphorylation, oxidative stress mediated through copper complexation, brain insulin resistance/signalling and mitochondrial toxicity. The original (fibril-centric) amyloid cascade hypothesis was reformulated when diffusible Aβ42 oligomers emerged as the more toxic species. To test whether the pre-fibrillary stabilized structure of peptide (2) exhibited an increase in cytotoxicity, the effect of dosing PC12 cells with varying concentrations of wild-type peptide (1) and the E22e peptide (2) was monitored. Addition of either peptide resulted in a reduction in cellular viability as determined by the reagent WST-1. At 20 μM, a 30% reduction in cellular viability was observed when dosing with wild-type peptide (1). Addition of the same concentration of the E22e peptide (2) resulted in an 80% reduction in viability (FIG. 10). The cellular viability of the PC12 cells was also found to be lower when peptide (2) was dosed at 10 μM, with close to a 65% reduction in cellular viability observed for peptide (2), compared with a 20% reduction when dosing with the same concentration of peptide (1) (FIG. 11). Pre-incubation (2 h or 4 h) of the peptides prior to dosing did not affect cytotoxicity (FIG. 12). The E22e variant offers a unique way of trapping an advanced aggregation intermediate of Aβ42 with enhanced toxicity, and highlights how a subtle structural change—a single chiral substitution—can have profound effects on aggregation and neurotoxicity.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A peptide comprising the amino acid sequence set forth in SEQ ID NO: 1 comprising one or more chiral substitutions at an electrostatic cluster amino acid residue selected from E22, D23, S26, K28, or any combination thereof, and wherein the peptide is no more than 49 amino acids in length.

2. The peptide of claim 1 consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14.

3. The peptide of claim 1, comprising a chiral substitution at E22.

4. The peptide of claim 3, comprising no other chiral substitutions.

5. The peptide of claim 1, wherein the peptide further comprises a non-chiral mutation.

6. The peptide of claim 5, wherein the peptide comprises one or more non-chiral mutations selected from: a non-chiral amino acid substitution, an amino acid deletion, an amino acid insertion, and any combination thereof.

7. The peptide of claim 6, wherein the non-chiral mutation is a familial Aβ peptide mutation.

8. The peptide of claim 7, wherein the peptide does not comprise a chiral substitution at amino acid residue D23, and the familial Aβ peptide mutation is a D23N mutation.

9. A method of screening a test agent for the ability to disrupt soluble oligomers of amyloid β (Aβ) peptides or the formation thereof, comprising:
incubating Aβ peptides comprising the peptide of claim 1 under Aβ peptide aggregation conditions;
contacting the Aβ peptides with a test agent; and
evaluating the presence or formation of soluble oligomers of Aβ peptides to determine whether the test agent has the ability to disrupt soluble oligomers of Aβ peptides or the formation thereof.

10. The method according to claim 9, wherein the Aβ peptides are contacted with the test agent subsequent to a period during which aggregation into soluble oligomers occurs, to determine whether the test agent has the ability to disrupt soluble oligomers of Aβ peptides.

11. The method according to claim 10, wherein determining whether the test agent has the ability to disrupt soluble oligomers of Aβ peptides comprises one or more of determining whether the soluble oligomers have transitioned to fibrillary aggregates, or determining whether the soluble oligomers have reverted to monomeric Aβ peptides.

12. The method according to claim 9, wherein the Aβ peptides are contacted with the test agent before or within a period during which aggregation into soluble oligomers would occur, to determine whether the test agent has the ability to disrupt the formation of soluble oligomers of Aβ peptides.

13. The method according to claim 9 where the test agent comprises an antibody or a small molecule.

14. The method according to claim 9 comprising screening a library of test agents for the ability to disrupt soluble oligomers of Aβ peptides or the formation thereof.

15. A method, comprising:
contacting a cell population with the peptide of claim 1;
after the contacting of the cell population with the peptide of claim 1, evaluating a parameter of the cell population selected from the group consisting of: cell viability, gene expression, ubiquitin-proteosome system (UPS) function, autophagy, and any combination thereof;
contacting the cell population with a second agent; and
after contacting the cell population with the second agent, evaluating a modulation by the second agent of a parameter of the cell population selected from the group consisting of: cell viability, gene expression, ubiquitin-proteosome system (UPS) function, autophagy, and any combination thereof.

16. The method of claim 15 where the second agent comprises a test agent.

17. The method of claim 15 where the cell population comprises neuronal cells.

18. The method of claim 17 where the cell population comprises pheochromocytoma cells.


SEQUENCE LISTING (14 sequences)

SEQ ID NO: 1 - protein, 36 aa, Homo sapiens
DAEFRHDSGYEVHHQKLVFFAEDVGSNKGAIIGLMV

SEQ ID NO: 2 - protein, 37 aa, Homo sapiens
DAEFRHDSGYEVHHQKLVFFAEDVGSNKGAIIGLMVG

SEQ ID NO: 3 - protein, 38 aa, Homo sapiens
DAEFRHDSGYEVHHQKLVFFAEDVGSNKGAIIGLMVGG

SEQ ID NO: 4 - protein, 39 aa, Homo sapiens
DAEFRHDSGYEVHHQKLVFFAEDVGSNKGAIIGLMVGGV

SEQ ID NO: 5 - protein, 40 aa, Homo sapiens
DAEFRHDSGYEVHHQKLVFFAEDVGSNKGAIIGLMVGGVV

SEQ ID NO: 6 - protein, 41 aa, Homo sapiens
DAEFRHDSGYEVHHQKLVFFAEDVGSNKGAIIGLMVGGVVI

SEQ ID NO: 7 - protein, 42 aa, Homo sapiens [sequence omitted]

SEQ ID NO: 8 - protein, 43 aa, Homo sapiens
DAEFRHDSGYEVHHQKLVFFAEDVGSNKGAIIGLMVGGVVIAT

SEQ ID NO: 9 - protein, 44 aa, Homo sapiens
DAEFRHDSGYEVHHQKLVFFAEDVGSNKGAIIGLMVGGVVIATV

SEQ ID NO: 10 - protein, 45 aa, Homo sapiens
DAEFRHDSGYEVHHQKLVFFAEDVGSNKGAIIGLMVGGVVIATVI

SEQ ID NO: 11 - protein, 46 aa, Homo sapiens
DAEFRHDSGYEVHHQKLVFFAEDVGSNKGAIIGLMVGGVVIATVIV

SEQ ID NO: 12 - protein, 47 aa, Homo sapiens
DAEFRHDSGYEVHHQKLVFFAEDVGSNKGAIIGLMVGGVVIATVIVI

SEQ ID NO: 13 - protein, 48 aa, Homo sapiens
DAEFRHDSGYEVHHQKLVFFAEDVGSNKGAIIGLMVGGVVIATVIVIT

SEQ ID NO: 14 - protein, 49 aa, Homo sapiens
DAEFRHDSGYEVHHQKLVFFAEDVGSNKGAIIGLMVGGVVIATVIVITL